(19) United States
(12) Patent Application Publication
(10) Pub. No.: US 2003/0129661 A1
(43) Pub. Date: Jul. 10, 2003

(54) Compositions, methods and kits for detection and quantification of plant and animal source contaminants

(76) Inventor: Lars Otto Uttenthal, Copenhagen East (DK)

Correspondence Address: LICATLA & TYRRELL P.C., 66 E. MAIN STREET, MARLTON, NJ, 08053, US

(21) Appl. No.: 10/319,280

(22) Filed: Dec. 12, 2002

Related U.S. Application Data
(60) Provisional application No. 60/340,753, filed on Dec. 18, 2001.

(51) Int. Cl. G01N 33/53

(52) U.S. Cl. 435/7.1

(57) ABSTRACT

Polyclonal and monoclonal antibodies are provided which are raised against a mixture of denatured animal or plant components in material derived from a single species. Also provided are methods and kits for use of these antibodies to detect and quantify materials derived from tissues of different animal or plant species in animal fodder of animal and/or plant origin, or in similar materials used, for example, as fertilizers or as ingredients in the production of foods and pharmaceuticals.

### INTRODUCTION

This application claims the benefit of priority from U.S. Provisional Application Serial No. 60/340,753, filed Dec. 12, 2001, which is herein incorporated by reference in its entirety.

### FIELD OF THE INVENTION

The present invention relates to compositions, methods and kits for detecting and quantifying materials derived from tissues of different animal or plant species in animal fodder of animal and/or plant origin, in similar materials used as fertilizers, and in preparations of animal and/or plant origin used as ingredients in products and processes in the food and pharmaceutical industries. The compositions, methods and kits of the present invention are particularly useful in the detection of bovine or other ruminant materials capable of transmitting prion diseases. Further, these compositions, methods and kits are useful for the avoidance of “cannibalism” in the feeding of livestock, including fish. While the compositions, methods and kits of the present invention can be routinely adapted for automated or semi-automated analysis, they can also be used at local testing sites and farms to detect contamination of animal feeds and other similar materials.

### BACKGROUND OF THE INVENTION

Methods for detecting contaminants in animal feeds have focused on detecting contamination of fish or plant-derived meals with material derived from terrestrial vertebrates.

In one method, sediment of the meal is subjected to optical microscopy to identify fragments of mammalian or avian bone. However, microscopy requires an experienced operator to detect the bone fragments. Further, it is impossible to identify the species from which the bone fragments are derived. Detection of 0.1% contamination of fishmeal with mammalian meat and bone meal is possible, but takes an average of 30 minutes.

In another class of methods, immunochemical techniques such as sandwich enzyme-linked immunosorbent assays (ELISAs) using existing antibodies against native proteins are used to detect bovine or porcine proteins that are believed to be resistant to heat denaturation. However, these methods are based on using antibodies to native proteins of animal origin, which are only present in minute amounts in heat-treated feeds. Thus, these methods do not provide sufficient sensitivity. Further, some of these methods exhibit a considerable background of non-specific reactivity, thus resulting in inadequate performance in validation tests.

A method is also currently being developed wherein the material is subjected to near-infrared (NIR) absorption spectroscopy. However, NIR absorption spectroscopy requires expensive equipment and skilled operators in a central laboratory. Further, this technique cannot differentiate between materials from different mammalian species. So far the technique can detect 3% contamination of fishmeal with mammalian meat and bone meal, but might be optimized to detect 1% contamination.

Another method under development is the use of the polymerase chain reaction (PCR) to detect and identify mitochondrial DNA from different animal and plant species. This method is potentially applicable to the detection of a wide range of animal and plant species in animal feeds or fertilizers and has been adapted from similar PCR methods developed for analyzing cooked food for human consumption. However, it is still not clear how effective this method will be with materials heated to over 130° C., when the mitochondrial DNA analyzed may break up into smaller and less species-specific fragments. Further, this method may prove too sensitive, e.g. detecting accidental trace contamination of no importance to human or animal health.

### SUMMARY OF THE INVENTION

An object of the present invention is to provide antibodies raised against total extractable denatured components (primarily proteins) of meals from different animal and/or plant species. Antibodies of the present invention may be polyclonal or monoclonal. Monoclonal antibodies of the present invention are selected either for specificity to a particular species or group of species, or for cross-reactivity with a wide group of species.

Another object of the present invention is to provide methods for producing a series of antibodies specific for heat-denatured proteins of a contaminating animal or plant species which comprises absorbing polyclonal antibodies raised against total extractable denatured components of meals with the extracted proteins from all species that are not to be detected in a given analytical procedure to remove undesired cross-reactivities. Antibodies produced in accordance with this method can then be used to detect the contamination of animal feeds from a variety of sources by materials from individual animal and/or plant species.

Another object of the present invention is to provide methods for use of these polyclonal and monoclonal antibodies to detect material from any group or combination of animal species, e.g. ruminants, in the presence of an excess of material derived from other sources, be they other animal species and/or plant species.

Yet another object of the present invention is to provide kits for detection of a material from any group or combination of animal species, e.g. ruminants, in the presence of an excess of material derived from other sources, be they other animal species and/or plant species. These kits comprise an antibody raised against total extractable denatured components (primarily proteins) of meals from different animal and plant species. In a preferred embodiment, the kit comprises additional components for extraction as well as additional components for conducting an immunoassay such as an ELISA or dipstick assay.

### BRIEF DESCRIPTION OF THE FIGURES

FIG. 1 is a graph showing the results of an inhibition ELISA performed according to the present invention. The antibody was a polyclonal rabbit antibody raised against the total extractable proteins from a heat-treated bovine meat and bone meal. The polyclonal antibody was absorbed with the total extractable proteins of fishmeal. It was preincubated at a final dilution of 1:10,000 against serial doubling dilutions of fishmeal extract (blocked squares) and against serial doubling dilutions of bovine meat and bone meal extract (blocked diamonds). The incubates were then transferred to microwells of an ELISA plate coated with bovine meat and bone meal extract at 1:1000. The binding of rabbit antibody to the coat was detected by an anti-rabbit Ig secondary antibody conjugated with horseradish peroxidase, and bound peroxidase activity was revealed by incubation with a color-forming substrate (ortho-phenylene diamine, or OPD), giving the optical density readings shown (signal).

FIG. 2 is a graph showing the results of another inhibition ELISA performed according to the present invention. Microwells of an ELISA plate were coated with bovine meat and bone meal extract at 1:200. Volumes of 50 μL of ⅕ dilutions of calibrator solutions containing respectively 0%, 0.1%, 1%, 2%, 5%, 10%, 20% and 50% extract of bovine meat and bone meal in extract of porcine meat and bone meal were added to the wells in triplicate, as were 50 μL volumes of dilutions of extracts of the meat and bone meal samples to be analyzed. The antibody was a polyclonal rabbit antibody raised against the total extractable proteins from a heat-treated bovine meat and bone meal and preabsorbed with the total extractable proteins of heat-treated porcine meat and bone meal. Volumes of 50 μL of this antibody at a dilution of 1/100 were added to each well. After incubation at room temperature for 1 hour, the binding of rabbit antibody to the coat was detected by an anti-rabbit Ig secondary antibody conjugated with horseradish peroxidase, and bound peroxidase activity was revealed by incubation with OPD, giving the standard curve shown. The values on the y scale were calculated by expressing the mean optical density value corresponding to a given contamination with bovine material as a percentage of the mean optical density corresponding to zero contamination. The horizontal line represents the detection limit for values differing from the mean optical density for zero contamination with 95% confidence. This corresponds to less than 0.1% contamination with bovine material.

FIG. 3 shows the inhibition of a mouse monoclonal antibody raised against heat-treated bovine albumin by extracts of fishmeal and bovine meat and bone meal, respectively. The protocol used to generate results of FIG. 3 was similar to that described in FIG. 1, except that a secondary antibody conjugate reacting with mouse immunoglobulins was used to detect bound primary antibody. It is seen that concentrations of fishmeal extract over 1000-fold (2E10) higher than those of bovine meat and bone meal extract are needed to initiate inhibition, indicating that the cross-reactivity of this antibody with fish material is less than 0.1%. As in FIG. 1, the concentration of fishmeal extract needed to produce a minor degree of inhibition is so high (dilutions of 1/10 and 1/20) that the effect may be due to the non-specific effect of a high protein concentration rather than to immunological cross-reactivity.

### DETAILED DESCRIPTION OF THE INVENTION

Whereas prior art has used antibodies raised against animal proteins in their native state, and has attempted to use these antibodies to detect such individual animal proteins as are expected to be resistant to the heat denaturation that takes place during the preparation of meat and bone meal and fish meal, the present method employs the strategy of raising polyclonal antibodies to the sum total of heat-denatured proteins extractable from meat and bone meal, fish meal or plant meals derived from different species. During their preparation these meals are subjected to heat treatment of varying temperature and duration under either wet or dry conditions. As a result, the animal proteins in them undergo a process of unfolding, whereby their original (“native”) structure is lost, and a variety of new, disordered but stable structures are exposed or created. This process is known as “denaturation”.

Denatured proteins will not react with antibodies that are directed against those parts of the native structure of the proteins that are destroyed during this process. In the denatured state, the proteins display a large number of new structures resulting from the unfolding process. Antibodies raised against these newly created structures will not react with the native protein, but may react with other denatured proteins, as the unfolding process may expose or create similar structures in different proteins. Antibodies raised against denatured proteins therefore cross-react with other denatured proteins.

The new structures exposed in the denatured proteins against which antibodies can be raised are often called “linear epitopes”, insofar as they consist of unfolded regions of the protein chain. The chances that such linear epitopes, consisting of no more than 4 to 6 amino-acid residues, are similar in different denatured proteins are very high, and in consequence it has not hitherto been regarded as practicable to raise antibodies capable of distinguishing between denatured proteins from different animal species.

In the present invention, however, it is demonstrated that such antibodies are indeed formed. They simply constitute a small minority of the sum total of the antibodies formed and the specificity of this minority of antibodies is hidden by the cross-reactivity of the vast majority of the other antibodies formed. However, as shown herein, when the cross-reacting antibodies are removed by well-known technique of absorbing them out with antigens from the species that are not to be analyzed, the species specificity of the remaining antibodies is revealed.

The presence of these species-specific antibodies is attributed in part to the violent nature of the denaturation process during the preparation of animal feeds, which promotes secondary reactions between side chains of the amino acids in the denatured proteins and between these side chains and other organic compounds present in the feed, generating new structures which may be species-specific. Some of these new structures may even be specific to different heat treatments of the same material, making it possible to demonstrate differences between the reactivities of antibodies raised against the same material subjected to different intensities of heat treatment.

In the present invention, polyclonal antibodies were raised against the extractable denatured proteins of meals derived from different animal and plant species. As stated above, these antibodies show a high degree of cross-reactivity with the denatured proteins extractable from meals derived from other species. However, they can be made specific for the denatured proteins from a given species by absorption with the extractable denatured proteins from all other animal and plant species that are relevant to the production of animal feeds. The number of animal species that must be taken into account to cover the vast majority of meat and bone meal production is limited to domestic varieties of ox, sheep and goat (to cover ruminant species considered to present an actual or potential risk of transmitting prion disease), possibly incorporating deer as venison production increases; pig; and domestic fowl including hen, turkey and possibly ostrich. Fishmeal is prepared from fish of various species, a widely different selection of species being used in different parts of the world. In this respect, the antibodies to be used to detect non-fish animal material in fishmeal are absorbed with extracts of fishmeal from many different sources in the northern and southern hemispheres. Plant meals are derived from a wide variety of species including soybeans, and many other beans, pulses, grains, seeds, and leaf and stem materials.

The general strategy for production of these polyclonal antibodies is to extract the meals derived from the individual species or group of species from which material is to be detected by a method optimized for solubilizing denatured proteins. The extracted protein mixture from each type of meal is then used as the antigen for raising polyclonal antibodies in animals such as rabbits or other mammalian or avian species suitable for this purpose. Antibodies are raised against the extracted denatured proteins from each species from which material is to be detected. The polyclonal antibodies are selected for optimum titer against the corresponding antigen preparation. The antibodies are then absorbed with the extracted proteins from all species that are not to be detected in a given analytical procedure. The absorption can be carried out by adding the protein solutions to the antibody or dilutions of antibody before use. Alternatively, absorption can be carried out by linking the proteins to a solid phase of a chromatographic column and passing the antibody solution through this column. The procedure is repeated for each species for which it is desired to remove cross-reactivity. Antibodies can be mixed to enable material from several species to be detected efficiently; the mixture is then absorbed with extracts of material from all species that are not to be detected.

Exemplary strategies for removing cross-reactivity are set forth below. As will be understood by those of skill in the art upon reading this disclosure, however, the following serves merely as examples and is in no way limiting to the various alternative strategies for removing cross-reactivity that can be routinely used in accordance with the teachings herein.

In one embodiment, wherein specific detection of bovine material in porcine meat and bone meal is desired, the antibody is raised against bovine material and absorbed with porcine, ovine and any other material for which detection is undesired.

In another embodiment, wherein specific detection of ruminant material in porcine meat and bone meal is desired, antibodies raised against materials from the relevant ruminant species are mixed and absorbed with porcine material.

In another embodiment, wherein specific detection of mammalian and avian material in fishmeal is desired, antibodies raised against materials from the relevant species are mixed and absorbed with material extracted from a variety of fishmeals.

In another embodiment, wherein specific detection of animal material in plant meals is desired, antibodies raised against materials from mammalian, avian and fishmeals are mixed and absorbed with materials extracted from a variety of plant meals.

In this way contaminating material from any combination of species can be detected in meals derived from other animal and/or plant species. Although the cross-reactivity displayed by the antibodies makes it possible to use the same antibody to detect materials from different species of the same class, once it has been appropriately absorbed (e.g. an antibody raised against bovine material can be used to detect ovine and porcine material), the efficiency with which material from different but related species is detected is less, and maximal sensitivity is obtained by using the antibody raised against material from the species that is to be detected.

The antibodies thus raised and absorbed can be used in immunoassays of a wide variety of designs to analyze meat and bone meals as well as fish and plant meals from different species for contamination with material from any other sufficiently different species.

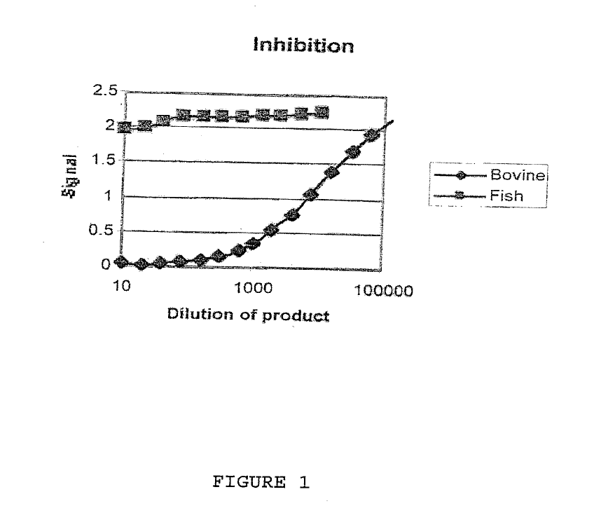

For example, FIG. 1 provides results from an inhibition ELISA using a polyclonal rabbit antibody raised against the total extractable proteins from a heat-treated bovine meat and bone meal. In this immunoassay, the polyclonal antibody was first absorbed with the total extractable proteins of fishmeal. The polyclonal antibody was then preincubated at a final dilution of 1:10,000 against serial doubling dilutions of fishmeal extract or bovine meat and bone meal extract. These pre-incubates were then transferred to microwells of an ELISA plate coated with bovine meat and bone meal extract at 1:1000 and binding of the polyclonal antibody to the coated microwells was detected using a detectably labeled anti-rabbit Ig secondary antibody. As can be seen in FIG. 1, the fishmeal extract did not inhibit binding of the antibody to the bovine meat and bone meal extract coating. The small inhibition seen at dilutions of 1:10, 1:20 and 1:40 of fishmeal extract is due to a protein mass effect (i.e. a high overall protein concentration) and is not specific. However, the bovine meat and bone extract significantly inhibited antibody binding even at dilutions as high as 1:40,960 in this experiment. Accordingly, as shown be this experiment, the polyclonal antibodies and methods of the present invention are capable of detecting the addition of 1 part of bovine meat and bone meal to 4096 parts of fish meal, i.e. 0.024%, even without optimization. This experiment also demonstrates that the denatured bovine material contains unique epitopes whose corresponding antibodies cannot be removed from the polyclonal antibody by absorption with an excess of fish meal extract, i.e. these epitopes are not found in denatured fish meal, and thus permit contamination of fish meal with bovine material to be detected.

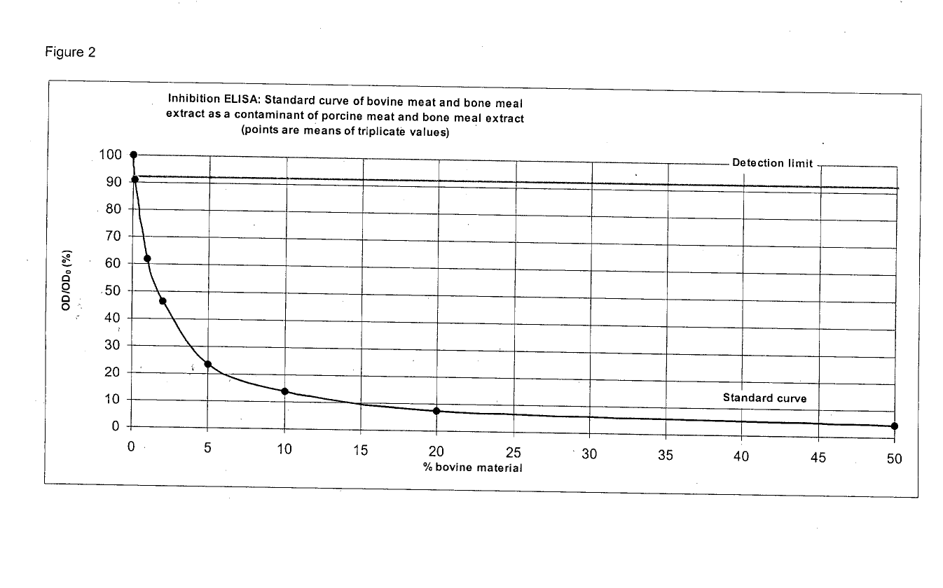

FIG. 2 provides results from a second inhibition ELISA performed using the compositions and methods of the present invention. In this immunoassay, microwells of an ELISA plate were coated with bovine meat and bone meal extract. Equal volumes of calibrator solutions containing increasing percentages of extract of bovine meat and bone meal in extract of porcine meat and bone meal and dilutions of extracts of meat and bone meal samples to be analyzed were then added to successive wells. A polyclonal rabbit antibody raised against the total extractable proteins from a heat-treated bovine meat and bone meal and preabsorbed with the total extractable proteins of heat-treated porcine meat and bone meal was then added to each well. Following incubation at room temperature for approximately one hour, the binding of polyclonal rabbit antibody to the wells was determined using a detectably labeled anti-rabbit Ig secondary antibody. Using this assay, a sample of porcine meat and bone meal from a factory known to receive both bovine and porcine slaughterhouse residues gave a reading from the standard curve corresponding to a 1.2% content of bovine material, whereas a sample of porcine meat and bone meal from a factory that does not receive bovine or ruminant slaughterhouse residues gave a reading corresponding to zero content of bovine material.

Monoclonal antibodies can also be prepared against epitopes on the denatured proteins present in the meal extracts. These monoclonal antibodies can be selected either to be specific for a particular species or group of species, or to be universally reactive with an epitope common to a wide group of species. The use of a monoclonal antibody specific to a particular species or group of species permits the design of a much wider range of analytical methods and may improve the sensitivity of such methods. The use of a monoclonal antibody that reacts with an epitope common to a wide group of species will also broaden the range of possible analytical methods; for example, such an antibody can be used as a detection antibody in a sandwich ELISA in which a coat of specifically generated and absorbed polyclonal antibody specifically captures material from the species to be detected.

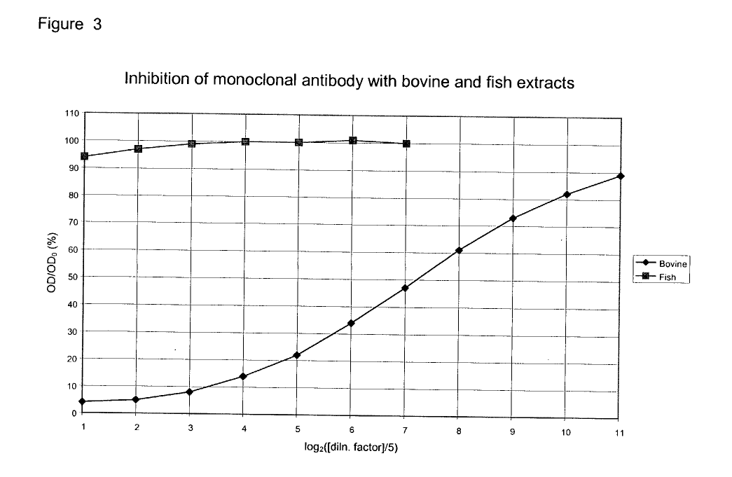

FIG. 3 shows the inhibition of a mouse monoclonal antibody raised against heat-treated bovine albumin by extracts of fishmeal and bovine meat and bone meal, respectively. The protocol used to generate results of FIG. 3 was similar to that described in FIG. 1, except that a secondary antibody conjugate reacting with mouse immunoglobulins was used to detect bound primary antibody. Concentrations of fishmeal extract over 1000-fold (2E10) higher than those of bovine meat and bone meal extract were needed to initiate inhibition, indicating that the cross-reactivity of this antibody with fish material is less than 0.1%. Thus, like the results shown for polyclonal antibodies of the present invention in FIG. 1, the concentration of fish meal extract needed to produce a minor degree of inhibition for the monoclonal antibody is so high (dilutions of 1/10 and 1/20) that the effect is likely due to the nonspecific effect of a high protein concentration rather than to immunological cross-reactivity. Accordingly, FIG. 3 provides an example of a monoclonal antibody that can be used to analyze the contamination of fishmeal with bovine material.

Accordingly, the present invention provides compositions comprising polyclonal antibodies raised against total extractable denatured components (primarily proteins) of meals from different animal and plant species and absorbed with the extracted proteins from all species that are not to be detected in a given analytical procedure to remove undesired cross-reactivities. The present invention also provides compositions comprising monoclonal antibodies raised against total extractable components (primarily proteins) of meals from different animal and plant species denatured. These antibody-containing compositions are useful in methods for detection of materials from any group or combination of animal species, e.g. ruminants, in the presence of an excess of material derived from other sources, be they other animal species and/or plant species. The antibody containing compositions of the present invention are particularly useful in detecting contamination of animal feeds from a variety of sources by materials from individual animal and/or plant species. Thus, the compositions and methods are useful in the detection of adulteration or contamination of permitted feeds with material of bovine or other ruminant origin, which may potentially spread prion diseases, and the detection of material derived from the species for which the feed is intended, to avoid “cannibalism”, which may promote the generation and spread of new prion or other infectious diseases. The compositions and methods of the present invention can also be applied to ingredients such as peptones and protein-rich preparations used in the food and pharmaceutical industries and in cell culture.

The compositions and methods of the present invention can be adapted for use in modern apparatus of sophisticated design to allow for rapid and sensitive analysis by automated or semi-automated techniques. However, the compositions and methods can also be adapted for use by non-specialized technical staff at local testing sites or even on farms to detect contamination of fodder or fertilizers with undesirable or illegal materials.

In the methods of the present invention, a contaminating animal or plant species can be detected in the presence of an excess of materials derived from other animal or plant species by contacting an extract of the materials with antibodies of the present invention and detecting any bound antibody. In some immunoassay formats, binding of the antibody to the extract is detected directly and is indicative of the presence of the contaminating animal or plant species. Alternatively, for immunoassay formats such as inhibition ELISA, antibody binding to a contaminant is determined indirectly by preventing the antibody from binding to a coated microwell plate. Thus, in this embodiment, inhibition of antibody binding is indicative of the presence of the contaminating animal or plant species.

The present invention also provides kits comprising an antibody of the present invention for detection of a material from any group or combination of animal species, e.g. ruminants, in the presence of an excess of material derived from other sources, be they other animal species and/or plant species. A preferred embodiment of the present invention comprises an ELISA or dipstick assay kit, containing, in addition to the antibody, an extraction solution for extracting the sample to be analyzed and other reagents and materials corresponding to the particular immunoassay embodiment.

For example, in the ELISA embodiment, the kit may further comprise an ELISA plate with microwells precoated with an extract of meat and bone meal from the species to be detected. To conduct this immunoassay, a dilution of the extracted sample is added, followed by addition of a dilution of a polyclonal antibody of the present invention that has been raised against material from the species to be detected and absorbed with materials from all the species not to be detected. The plate is then incubated at room temperature. The wells of the plate are washed, an enzyme-labeled secondary antibody is added, and the plate is incubated. Following the incubation, the wells of the plate are washed again, a color-forming substrate solution is added, and the plate is incubated again. The optical density of the color formed in the wells of the plate is then read in a microplate reader. Accordingly, a kit for conducting this ELISA preferably comprises a plate with microwells precoated with material from the species to be detected, a dilution of primary antibody against this material, said antibody having been preabsorbed with materials from species that are not to be detected, a dilution of the secondary antibody against immunoglobulins of the species from which the primary antibody was obtained, the secondary antibody being conjugated with an enzyme, a solution of color-forming substrate for this enzyme, a negative control solution containing a dilution of an extract of uncontaminated meal of the type to be analyzed, a positive control solution containing a dilution of an extract of the potentially contaminating material, and a series of standard solutions representing different degrees of contamination. The negative controls give a strong color, while the positive control gives little or no color. Increasing degrees of contamination reduce the color development in the wells.

In the dipstick embodiment, the primary antibody is incorporated into a dipstick carrying a color-forming mechanism well known to those skilled in the art. The dipstick which is preferably attached to the lid of a tube in which a dilution of sample extract is placed, is dipped in the solution by screwing on the lid, and a color forms on it if the solution contains material from the species from which contamination is to be detected. Other dipstick embodiments include, but are not limited to, use of colored microparticles coated with the primary antibody and incorporated into the lower end of a cellulose fiber strip. When the lower end of the strip is dipped into the extract to be tested, the beads travel up the strip by capillary action, and if they have picked up the antigen to be detected, they are arrested by a band of primary antibody incorporated into the cellulose at a higher level, thus forming a colored band. A further band of antibody against immunoglobulins of the species from which the primary antibody is derived is placed at an even higher level. This arrests the beads at that level whether or not they have picked up antigen, and the formation of a colored band at that level thus provides a control that the strip is functioning correctly.

The following nonlimiting examples are provided to further illustrate the present invention.

### EXAMPLES

### Example 1

### Materials

Meat and bone meals were prepared from cattle, pigs, sheep, goats, domestic fowl (chickens+turkeys) according to industrial methods reduced to a laboratory scale and subjected to heating to 133° C. or 150° C. or 159° C. for 20 minutes in either the water phase before reduction to fine granules or in the steam phase after reduction to granules. In addition, bovine albumin was subjected to heating under the same conditions. This provided 6 different materials each subjected to 6 different heat treatments, giving a total of 36 different preparations.

### Example 2

### Extraction

The preparations of Example 1 were extracted at set proportions of solid to liquid in aqueous buffers of different pH and ionic strength, with and without different detergents and with and without 6 M guanidine hydrochloride. The efficiency of extraction of proteins was determined by measuring the protein concentration of the extract by a dye binding method using Coomassie brilliant blue G.

### Example 3

### Polyclonal Antibody Preparation

An extract of each preparation was adsorbed onto aluminum hydroxide gel and used to immunize two New Zealand white rabbits per preparation. The gel suspension was emulsified in Freund's complete adjuvant for the first injection and in incomplete adjuvant for subsequent injections. Rabbits were bled 12 days after each injection and the sera characterized by ELISA in polystyrene wells coated with different extracts.

### Example 4

### Pooling and Absorption of Antisera

Selected sera were then pooled in various ways and absorbed with different combinations of extracts at different ratios of extract to serum according to the strategies outlined previously.

### Example 5

### Standards

Standards for use in analysis were prepared by adding extract from the species to be detected to extract of the other species present in the material to be analyzed.

### Example 6

### Coating of Microwells

For inhibition ELISA, polystyrene microwells were coated by standard methods with dilutions of extract from the species to be detected. The wells could be dried and stored in foil.

What is claimed is:

1. An antibody raised against total extractable denatured components of meals from different animal and plant species.

2. The antibody of claim 1 which is polyclonal and which is absorbed with extracted proteins from species in meal that are not to be detected by the antibody to remove undesired cross-reactivities.

3. The antibody of claim 1 which is monoclonal.

4. The monoclonal antibody of claim 3 which is specific to a particular animal or plant species or group of animal or plant species.

5. The monoclonal antibody of claim 3 which is cross-reactive with a group of animal or plant species.

6. A method for producing polyclonal antibodies specific for heat-denatured proteins of a contaminating animal or plant species comprising raising a polyclonal antibody against total extractable denatured components of meals from different animal and plant species and absorbing the polyclonal antibody with extracted proteins from species in the meals that are not to be detected by the antibody to remove undesired cross-reactivities and produce antibodies specific for heat-denatured proteins of a contaminating animal or plant species.

7. A method for detecting a contaminating animal or plant species in the presence of an excess of materials derived from other animal or plant species comprising contacting an extract of the materials with the antibody of any of claims 1 through 5 and detecting any bound antibody wherein detection of the bound antibody is indicative of the presence of the contaminating animal or plant species.

8. A method for detecting a contaminating animal or plant species in the presence of an excess of materials derived from other animal or plant species comprising contacting an extract of the materials with the antibody of any of claims 1 through 5 and detecting any bound antibody wherein inhibition of antibody binding to the extract is indicative of the presence of the contaminating animal or plant species.

9. A kit for detection of a contaminating animal or plant species in the presence of an excess of material derived from other animal or plant species comprising the antibody of any of claim 1 through 5.
